(19) United States
(12) Patent Application Publication
(10) Pub. No.: US 2024/0124192 A1
(43) Pub. Date: Apr. 18, 2024

(54) SYSTEMS, APPARATUSES, AND METHODS FOR STORING AND DISPOSING OF SHARP OR HAZARDOUS OBJECTS

(71) Applicant: Christopher O'Meara, Chico, CA (US)

(72) Inventor: Christopher O'Meara, Chico, CA (US)

(21) Appl. No.: 18/380,183

(22) Filed: Oct. 15, 2023

Related U.S. Application Data
(60) Provisional application No. 63/416,509, filed on Oct. 15, 2022.

(51) Int. Cl. B65D 25/10; A61B 50/36; B65D 43/16; B65D 43/22; B65D 53/02

(52) U.S. Cl. CPC B65D 25/103; A61B 50/362; B65D 25/102; B65D 43/16; B65D 43/22; B65D 53/02; B65D 2251/1058; B65D 2251/205; B65D 2543/00194; B65D 2543/00972

(57) ABSTRACT

Aspects of the present disclosure provide technologies for storing, transporting, and disposing of medical waste (e.g., sharps and biohazardous materials). Medical waste commonly results after medical treatment of patients. For example, a piece of medical equipment may be stored after use in a channel of a structural element and then restrained within the channel using a restrictive element. The entire assembly may be surrounded by an internal cavity formed by a first portion of a housing abutting a second portion of a housing, thereby creating a leak-resistant seal. The technologies described herein may be portable so that first responders and medical practitioners may carry them to any emergency location. In situations, the housing surrounding the assembly can be incinerated, thereby destroying any pathogenic contamination.

### CROSS-REFERENCE

The present application claims priority to U.S. Provisional Patent Application No. 63/416,509 filed Oct. 15, 2022, entitled, “SYSTEMS, APPARATUSES, AND METHODS FOR STORING AND DISPOSING OF SHARP OR HAZARDOUS OBJECTS”, which is entirely incorporated herein by reference for all purposes.

### BACKGROUND

A wide variety of devices and methods exist for storing used medical equipment (e.g., sharps) and biohazardous waste materials (e.g., blood, equipment contaminated with biological fluids) generated during the course of medical treatment of a patient in hospitals and clinics that are designed to prevent accidental human contact. A Non-limiting example of such objects include needles for diagnostic and treatment. The use of such devices becomes imperative after use of sharps (e.g., needles, syringes, hypodermic needles, lancets, and intravenous starts) because patients may carry pathogens.

Unfortunately, the existing devices are bulky leading to a variety of inefficiencies relating to use of space and sometimes lack effectiveness at preventing accidental contact with a first responder and/or medical practitioner. For example, there is currently no effective solution for storing and transporting used medical devices and biohazardous waste materials in non-hospital and non-clinical settings (e.g., from the location of an emergency or in other uncontrolled settings). This lack of effectiveness may lead to accidental contact of used medical equipment and biohazardous waste materials with the medical practitioner. Not only does this sometimes cause severe health consequences, but even if the accidental contact does not lead to transmission of a pathogen, time and costs associated with time away from work and regular testing have a negative impact on the people and communities involved along with associated costs. The field needs medical practitioners working to improve the health of our communities while minimizing risk to themselves.

As such, there is a need for technologies that effectively address these and other needs, by limiting the risk and costs associated with accidental contamination of medical practitioners. The present disclosure addresses these and other needs.

### SUMMARY

Aspects of the disclosure describe systems for safely storing sharp or hazardous materials, in accordance with various embodiments. In various embodiments, the system for safely storing sharp or hazardous materials comprises a first portion of a housing, a second portion of the housing detachably coupled to the first portion of the housing, and a structural element positioned within an internal cavity of the second portion.

In various embodiments, the structural element comprises a floor, a first wall projecting upwardly from the floor, and a second wall projecting upwardly from the floor, wherein the floor and the two walls partially enclose a channel.

In various embodiments, the first wall and the second wall each wall each comprise a thick region including a first end, an opposing second end, a height, and a width, and a first thin region including a first end, an opposing second end, a height, and a width, wherein the first end of the thick region is joined to the first end of the first thin region, wherein the height of the thick region is greater than the height of the first thin region, wherein the width of the thick region is greater than the width of the first thin region.

In various embodiments, the first wall and the second wall each wall each comprise a second thin region including a first end, a second end, a height, and a width, wherein the second end of the thick region is joined to the second end of the second thin region, wherein the height of the thick region is greater than the height of the second thin region, wherein the width of the thick region is greater than the width of the second thin region.

In various embodiments, the first wall and the second wall each wall each comprise a first end cap joined to the second end of the first thin region and a second end cap joined to the first end of the second thin region, wherein a height of the first end cap and a height of the second end cap are equal.

In various embodiments, each of the thin regions comprise an inner surface connecting an outer surface by a top surface, wherein the top surface is perpendicular to the inner surface and the outer surface and a curved surface connecting the inner surface to the outer surface, wherein the curved surface is located opposite to the top surface.

In various embodiments, the first wall and the second wall each wall each comprise a single and contiguous inner surface spanning the first end cap, the first thin region, the thick region, the second thin region, and the second end cap along a single plane.

In various embodiments, the first wall comprises a first recess and the second wall comprises a second recess.

In various embodiments, the system further comprises an elongated restrictive element, wherein the elongated restrictive element spans across the channel and a first portion of the elongated restrictive element is positioned within the first recess and a second portion of the elongated restrictive element is positioned within the second recess.

In various embodiments, the elongated restrictive element forms a contiguous loop and is in physical contact with a curved surface of the structural element, wherein the curved surface joins the restrictive element receiving surface to a second surface of the floor of the structural element, and wherein a first surface of the floor opposes the second surface and first and second walls are joined to the first surface.

In various embodiments, the elongated restrictive element comprises an elastic band. In various embodiments, the elongated restrictive element comprises a rigid material.

In various embodiments, the first and the second wall each comprise a planar element projecting upwardly from a first surface of the floor of the structural element, wherein the planar element is bounded by a perimeter comprising a bottom surface joined to the first surface of the floor, an upper surface joined to the bottom surface by two opposing outward surfaces, a lower surface of the first recess joined to the upper surface by two opposing side surfaces, and a lower surface of the second recess joined to the upper surface by two opposing side surfaces.

In various embodiments, the second portion of the housing further comprises a planar element having an interior surface and an opposing exterior surface bounded by a perimeter and a wall projecting away from the planar element and joined to the perimeter of the planar element.

In various embodiments, the first portion of the housing further comprises a planar element having an interior surface and an opposing exterior surface bounded by a perimeter and a wall projecting away from the planar element and joined to the perimeter of the planar element.

In various embodiments, the wall of the second portion comprises a thick region having an exterior surface and an interior surface, wherein the interior surface of the thick region meets an interior surface of the planar element at a seam, a thin region joined to the thick region, wherein the thin region has an exterior surface and shares the interior surface with the thick region, and a lip connecting the exterior surface of the thick region with the exterior surface of the thin region, wherein a top surface joins the interior surface with the exterior surface of the thin region.

In various embodiments, the wall of the first portion comprises a thick region having an exterior surface and an interior surface, wherein the interior surface of the thick region meets an interior surface of the planar element at a seam, a thin region joined to the thick region, wherein the thin region has an interior surface and shares the exterior surface with the thick region, and a lip connecting the interior surface of the thick region with the interior surface of the thin region, wherein a top surface joins the interior surface of the thin region with the exterior surface.

In various embodiments, the lip of the second portion abuts the top surface of the first portion and the exterior surface of the thin region of the second portion abuts the interior surface of the thin region of the first portion.

In various embodiments, the system further comprises a groove extends along the lip of the second portion, wherein a sealing element resides within the groove. In various embodiments, the sealing element comprises an O-ring.

In various embodiments, the structural element is coupled to the planar element of the second portion of the housing. In various embodiments, the structural element is coupled to the planar element of the second portion of the housing using one or more clips, a weld, or an adhesive.

In various embodiments, the system further comprises a securing element, wherein the securing element comprises a first element positioned on the first portion of the housing, wherein the first element comprises a locking surface and a second element positioned on the second portion of the housing, wherein the second element comprises a locking surface. In various embodiments, the system further comprises a removeable element for preventing the first element from interacting with the second element.

Aspects of the disclosure describe methods for safely storing sharp or hazardous materials, in accordance with various embodiments. In various embodiments, the method comprises depositing a medical device into a channel of a structural element, restricting movement of the medical device within the channel, and sealing the medical device and the structural element within a leak resistant housing. In various embodiments, the step of restricting movement further comprises positioning a restrictive element over the channel.

In various embodiments, the structural element comprises a floor, a first planar element protruding from the floor, wherein the first planar element comprises a first recess positioned along an upper surface of the first planar element, and a second planar element protruding from the floor, wherein the second planar element comprises a second recess positioned along an upper surface of the second planar element, wherein the first planar element is spaced apart from the second planar element and the floor and planar elements form the channel.

In various embodiments, the step of restricting movement further comprises contacting the restrictive element with the first recess and the second recess of the structural element. In various embodiments, the step of restricting movement further comprises contacting the restrictive element with a surface of the first planar element and a surface of the second planar element. In various embodiments, the step of restricting movement further comprises contacting the restrictive element with a restrictive element receiving surface of the first planar element and with a restrictive element receiving surface of the second planar element. In various embodiments, the step of restricting movement further comprises contacting the restrictive element with a curved surface of the first planar element and with a curved surface of the second planar element. In various embodiments, the step of restricting movement further comprises contacting the restrictive element with a second surface of the floor and the first and second protrusions are joined to an opposing first surface of the floor.

In various embodiments, the restrictive element comprises an elongated elastic portion and the method further comprises the steps of expanding the elongated elastic portion prior to the depositing step and contracting the elongated elastic portion during the restricting movement step.

In various embodiments, the step of sealing the medical device within a leak resistant housing, further comprises positioning a first portion of the housing adjacent to a second portion of the housing. In various embodiments, the step of positioning a first portion of the housing adjacent to a second portion of the housing, further comprises contacting a surface of a wall of the first portion with a surface of a wall of the second portion to completely enclose an internal cavity of the leak resistant housing. In various embodiments, the step of positioning the first portion of the housing adjacent to the second portion of the housing comprises actuating a hinge, wherein a first portion of the hinge is positioned on a surface of the first portion of the housing and a second portion of the hinge is positioned on a surface of the second portion of the housing.

In various embodiments, the method further comprises actuating a securing element to secure the first portion of the housing to the second portion of the housing, wherein a first element of the securing element is positioned on the first portion of the housing and a second element of the securing element is positioned on the second portion of the housing.

In various embodiments, the method further comprises incinerating the leak resistant housing.

### BRIEF DESCRIPTION OF THE DRAWINGS

FIG. 1 is a perspective view of an apparatus in an open position illustrating an internal cavity for storing and transporting sharp or hazardous objects in accordance with various embodiments.

FIG. 2 is a perspective view of an apparatus in an open position illustrating external surfaces of first and second portions for storing and transporting sharp or hazardous objects in accordance with various embodiments.

FIG. 3 is a perspective view of an apparatus for storing and transporting sharp or hazardous objects in a closed position in accordance with various embodiments.

FIG. 4 is a perspective view of an apparatus for storing and transporting sharp or hazardous objects in a closed position in accordance with various embodiments.

FIG. 5 is a perspective view of a first portion of a housing for an apparatus for storing and transporting sharp or hazardous objects in accordance with various embodiments.

FIG. 6 is a perspective view of a second portion of a housing for an apparatus for storing and transporting sharp or hazardous objects in accordance with various embodiments.

FIG. 7 is a cross-section view of a first portion and a second portion of a housing for an apparatus for storing and transporting sharp or hazardous objects in a closed position in accordance with various embodiments.

FIG. 8 is a perspective view of a structural element of an apparatus for storing and transporting sharp or hazardous objects in accordance with various embodiments.

FIG. 9 is a reverse perspective view of a structural element of an apparatus for storing and transporting sharp or hazardous objects in accordance with various embodiments.

FIG. 10 is an illustration of a removeable element positioned between a first element and a second element of a securing element in accordance with various embodiments.

FIG. 11 is an illustration of a wall of a structural element in accordance with various embodiments.

FIG. 12 is a flow chart illustrating a method of use for storing and transporting sharp or hazardous objects in accordance with various embodiments.

### CERTAIN DEFINITIONS

Connected: As used herein, the term “connected” and “coupled to” are used interchangeably and may refer to two or more objects being positioned adjacent to, abutting, or in physical contact with one another. Sometimes two connected objects may be a single object having two or more features positioned adjacent to or in physical contact with one another (e.g., a first surface touching a second surface). Objects may be connected as a single component or include two or more objects welded together. In some circumstances, objects can be affixed or joined using an intermediary. In some cases, two or more objects or materials may be positioned next to each other without being connected. In other cases, two or more objects may be positioned next to each other while being connected in a manner described herein or in the art. Non-limiting examples of intermediaries may include tacks, rivets, nails, clips, pins, screws, staples, etc. In some circumstances, objects can be affixed or joined using a weld.

First Responder: As used herein, the term “first responder,” may refer to a trained individual who arrives first or early to the scene of an emergency, treatment location, or critical incident and provides immediate medical treatment, assistance, care, and support to those affected including patients. First Responders may be tasked with ensuring the safety and well-being of patients, stabilizing the situation, health status of patients, and preparing for the arrival of medical and rescue personnel. First responders may include police officers, firefighters, emergency medical technicians (EMTs), paramedics, search and rescue teams, medical practitioners (e.g., as described herein), and other professionals who have the skills and training to address various types of emergencies including those requiring medical treatment of a patient.

Joined: As used herein, the term “joined,” may refer to two or more objects coupled to one another that were once separate, but now a contiguous component. Non-limiting examples of joining techniques may include welding, solvent bonding, and adhesion. Joined may refer to two or more features of a single object that were formed at the same time during a manufacturing process (e.g., injection molding).

Medical Equipment: As used herein, the terms “medical equipment” and “medical device” are used interchangeable and may refer to a variety of tools used in the medical field. Medical equipment may be used in the medical treatment of a patient and may include surgical tools, tools for administering medication, and tools for diagnosis. Non-limiting examples of medical equipment may include needles, razors, cutting devices, scalpels, or any object susceptible to being contaminated by a patient.

Medical Practitioner: As used herein, the term “medical practitioner” and “healthcare practitioner” are used interchangeably and may refer to a doctor, a nurse, a nurse practitioner, a medical assistant, an emergency medical technician, a firefighter, a first responder to the scene of an accident, or any other person using a medical device to treat a patient.

Medical Treatment: As used herein, the term “medical treatment,” may refer to the application of medication or use of medical tools on a patient.

Medical Waste: As used herein, the term “medical waste,” may refer to biohazards, infectious medical waste, and used medical equipment.

Pathogens: As used herein, the term “pathogen,” may refer to a microorganism that can cause a disease in a biological organism (e.g., a human or animal). Pathogens may be commonly found in and on medical waste. For example, patients or biological organisms may carry pathogens such as bacteria and viruses. During the course of medical treatment, medical equipment may be used that may be contaminated by pathogens originating from the patient or biological organism or the surrounding environment.

Patient: As used herein, the term “patient,” may refer to a human or animal being administered medical treatment. A biological organism may be a patient.

Treatment Location: As used herein, the term “treatment location,” may refer to any place where medical treatment may be administered. Non-limiting examples of treatment locations may include hospitals, clinics, and locations of emergencies. Locations of emergencies can be anywhere a patient may be present and in need of medical treatment. Non-limiting examples of common medical locations may include roadsides where automobile accidents have occurred, a location of any vehicle (e.g., plane, train, boat, bicycle, or other mode of transportation) accident, a residence of a patient, houses, sidewalks, homeless encampments, public areas, private areas, any outdoor location, any indoor location.

### DETAILED DESCRIPTION OF CERTAIN EMBODIMENTS

### Overview

Safety in the workplace may be particularly concerning for healthcare professionals due to the potential of being exposed to sick patients. Healthcare professionals are particularly at risk of being exposed to pathogens from medical waste. For example, exposure to sharp medical waste (e.g., needles) may be hazardous because it has the potential to expose medical practitioners to needlesticks while attending a patient at a treatment location. About 350,000 needlestick and other sharps related injuries occur each year to hospital-based healthcare professionals. Similar injuries occur while providing emergency care. Sharps injuries make a medical practitioner susceptible to transmission of hepatitis B virus (HBV), hepatitis C virus (HCV), and human immunodeficiency virus (HIV), and in the transmission of more than 20 other pathogens. See https://www.cdc.gov/sharpssafety, the disclosure of which is incorporated herein by reference in its entirety.

One of many issues in the field is the inability to safely dispose of medical waste and, in particular, to safely dispose of medical waste if the location of treatment is outside of a clinic or hospital. For example, currently no easy and effective storage, transportation, and/or disposal technologies exist for medical waste in the field (e.g., at the site of an accident or in a patient's home). The apparatuses, systems, and methods provided herein provide safe and effective ways to store, transport, and dispose of medical waste, thereby mitigating the risk to healthcare professionals. Such apparatuses, systems, and methods are particularly helpful in reducing the number of needlesticks per year for emergency responders, police department personnel, fire department personnel, military personnel, for civilian personnel, or for anyone at or near a treatment location as a user or a bystander.

### Exemplary Systems and Apparatuses

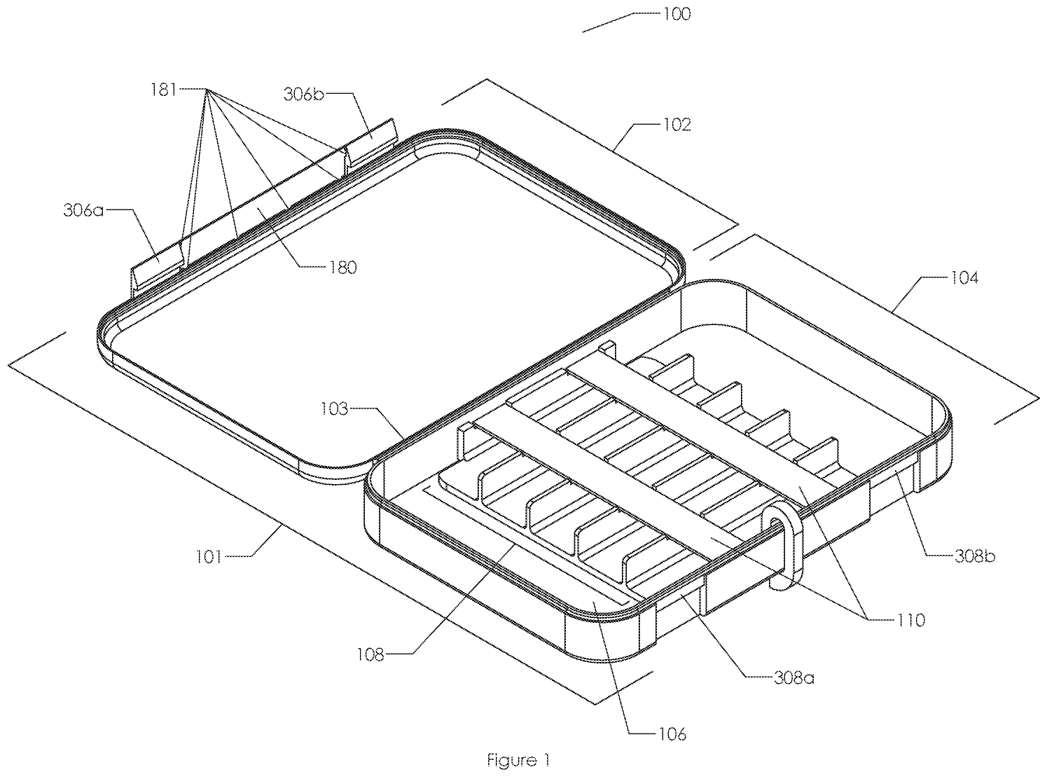

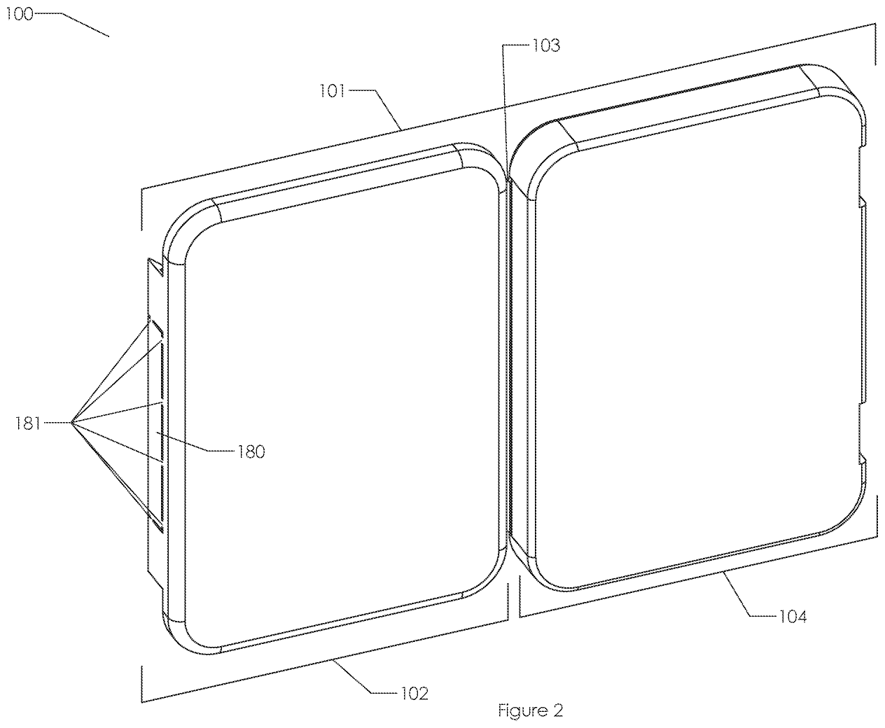

FIGS. 1 and 2 are perspective views of an apparatus in an open position in accordance with various embodiments. FIG. 1 illustrates a perspective view of the apparatus showing an internal cavity for storing and transporting sharp or hazardous objects 100 in accordance with various embodiments. FIG. 2 illustrates a perspective view of the apparatus showing external features of a lid and a chamber in accordance with various embodiments. In various embodiments, the systems and apparatuses described herein may be disposable. In various embodiments, the systems and apparatuses described herein may be single-use or be comprised of one or more single-use components (e.g., components within the internal cavity of the apparatus may be disposable). In various embodiments, the apparatus may be designed to be sealed (e.g., using one or more of the securing and/or latching features or elements described herein), thereby, preventing access to the internal cavity by a user. In various embodiments, the apparatus may be portable.

In various embodiments, an apparatus for storing and transporting sharp or hazardous objects 100 may comprise a housing 101. In many embodiments, the housing 101 may comprise a first portion 102 and a second portion 104. In various embodiments, the first portion 102 of the housing 101 may be designed to interact with the second portion 104 of the housing 101 to enclose an internal cavity 106. In various embodiments, the internal cavity 106 may be designed to hold or receive a structural element 108.

In various embodiments, a housing 101 may surround an internal cavity 106. For example, when a first portion 102 of the housing 101 and a second portion 104 of the housing 101 are connected, abutted, or fitted together to form a closed position (e.g., See FIG. 2) the first portion 102 and the second portion 104 may completely enclose an internal cavity 106. In other examples, when a first portion 102 and a second portion 104 are connected they may partially enclose an internal cavity 106. In various embodiments, an internal cavity 106 may be designed to be leak resistant or leak proof. As such, an apparatus for storing and transporting sharp or hazardous objects 100 may protect a user (e.g., a medical practitioner) from being exposed to dangerous medical equipment and/or medical waste. A common danger in the field may include medical and/or medical waste having been contaminated with a pathogen.

In various embodiments, a connector 103 may connect or join a first portion 102 of a housing 101 to a second portion 104 of a housing 101. In various embodiments, the connector 103 may comprise a flexible piece of material capable of deforming and returning to an original state (e.g., a living hinge). In various embodiments, connector 103 may be an integrated piece of the first portion 102 and the second portion 104. For example, in various embodiments, a housing 101 may comprise a single piece of molded material including the first portion 102, the second portion 104, and the connector 103.

An example of a connector 103, according to various embodiments may include a piece of molded plastic that may be continuous with the first portion 102 and the second portion 104 of the housing 101. In some embodiments, connector 103 may include a separate component from the first portion 102 and the second portion 104 of the housing 101. In many embodiments, the connector 103 may include an elastic component.

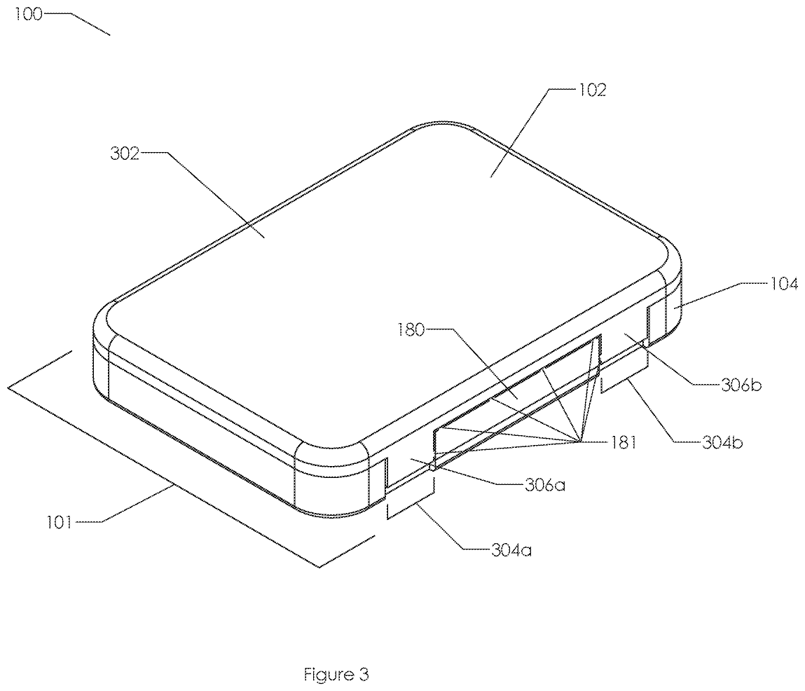

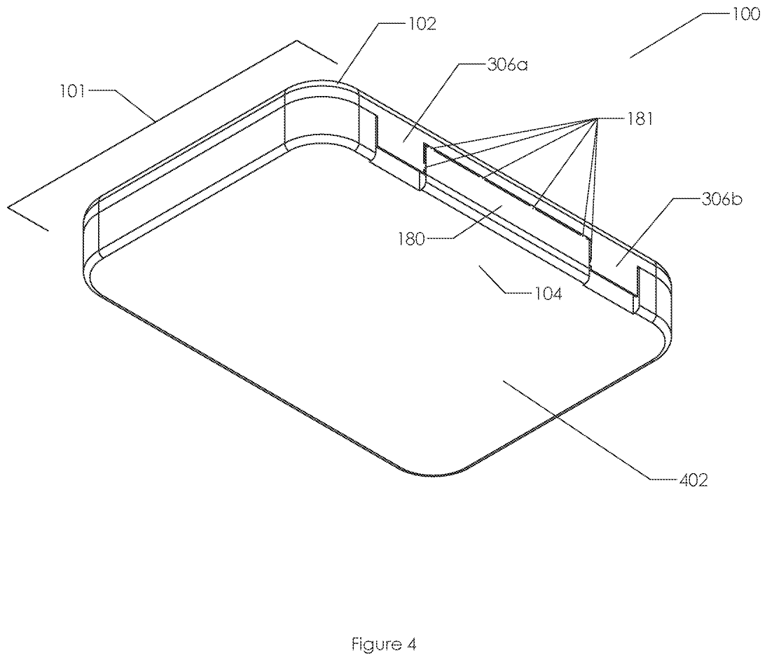

FIG. 3 is a perspective view of an apparatus for storing and transporting sharp or hazardous objects in a closed position in accordance with various embodiments. FIG. 4 is a perspective view from an opposing angle of the apparatus for storing and transporting sharp or hazardous objects in a closed position in accordance with various embodiments. In various embodiments, a connector 103 may provide mechanical movement of a first portion 102 of a housing 101 and a second portion 104 of the housing 101 so that the first portion 102 and the second portion 104 may move relative to one another while staying connected. Such mechanical movement may allow an apparatus for storing and transporting sharp or hazardous objects 100 to move from an open position (e.g., See FIGS. 1 and 2) to a closed position (e.g., See FIGS. 3 and 4) as well as any position in between (e.g., a partially closed position).

Some connectors 103 may include a moveable joint. In some embodiments, a connector 103 may include a hinge. In some embodiments, a connector 103 may include ball bearings. In some embodiments, a connector 103 may include one or more springs. In some embodiments, a connector 103 may include a barrel. In some embodiments, a connector 103 may include an overlay hinge. In some embodiments, a connector 103 may include an offset hinge. In some embodiments, a connector 103 may include a piano hinge. In some embodiments, a connector 103 may include a strap hinge. In some embodiments, a connector 103 may be include mechanical components that may be concealed.

Some connectors 103 may include latching or locking features alternatively or in addition to the securing elements described herein. Such latching or locking feature may allow a first portion 102 and a second portion 104 of a housing 101 to be positioned stably in an open position, a closed position, or a partially closed position. For example, a first element 306a, 306b of a securing element may interact with a second element 308a, 308b of a securing element to lock the first portion 102 of the housing 101 to the second portion 104 of the housing 101.

In some embodiments, a connector 103 may not be necessary. For example, a first portion 102 of a housing 101 and a second portion 104 of a housing may be completely separated while in an open position. In a closed position, the first portion 102 and the second portion 104 may connect to form a closed position without use of a connector 103. In some embodiments, the first portion 102 and the second portion 104 of the housing may be held together by a frictional force while in the closed position. For example, a surface of the first portion 102 may be tightly pressed against a surface of the second portion 104, thereby, preventing relative movement between the first portion 102 and the second portion 104 while the apparatus is in the closed position.

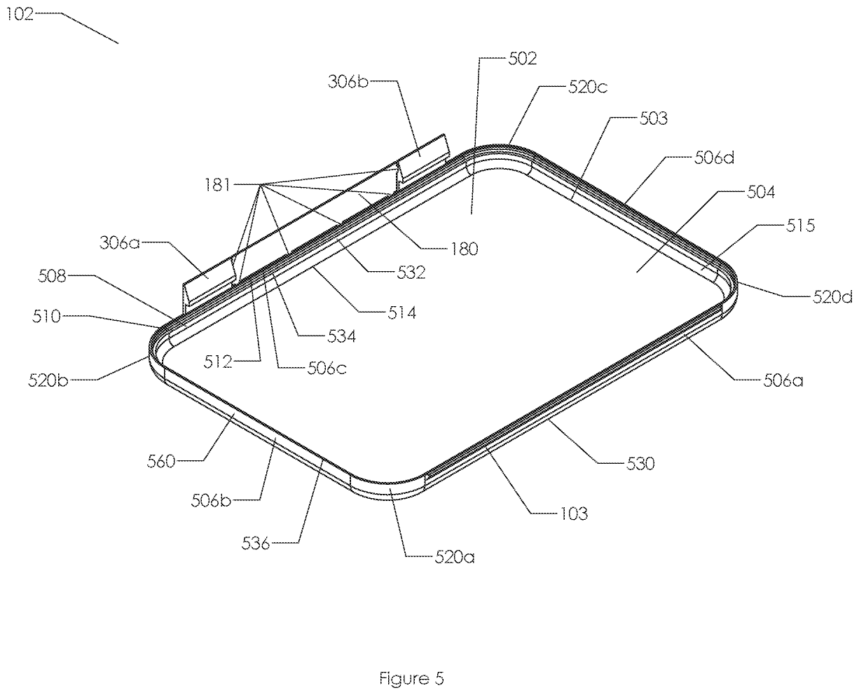

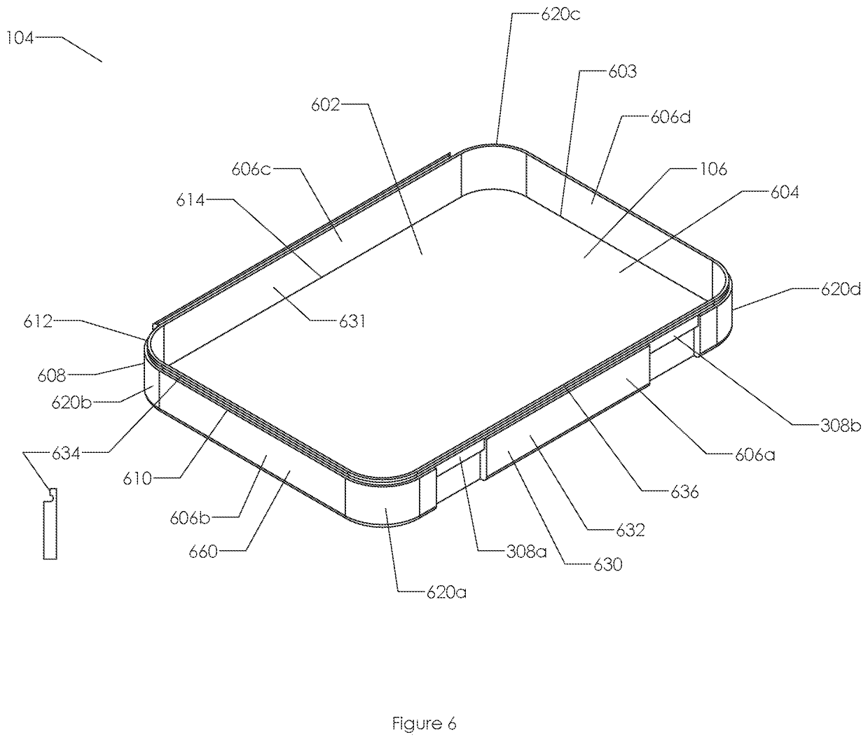

Now referring to FIG. 5, a perspective view of a first portion 102 of a housing 101 for an apparatus for storing and transporting sharp or hazardous objects is illustrated in accordance with various embodiments. In various embodiments, the first portion 102 can act as a lid to a second portion 104 (e.g., See FIG. 6) of the housing 101.

In various embodiments, a first portion 102 of a housing 101 may include a planar element 502 having an interior surface 504 and an opposing exterior surface 302 (e.g., See FIG. 3). In various embodiments, the planar element 502 may include a perimeter 503 encircling the planar element 502.

In various embodiment, a perimeter wall 560 may extend away from an interior surface 504 of a planar element 502. In various embodiments, the perimeter wall 560 may be positioned adjacent to and encircle or partially encircle a perimeter 503 of the planar surface 502. In various embodiments, the perimeter wall 560 may include one or more walls 506a, 506b, 506c, 506d and one or more corner elements 520a, 520b, 520c, 520d.

In various embodiments, a first portion 102 of a housing 101 may include one or more walls 506a, 506b, 506c, 506d extending away from an interior surface 504 of a planar element 502 of the housing 101. In various embodiments, the walls 506a, 506b, 506c, 506d may be positioned substantially adjacent or on a portion of a perimeter 503 of the planar surface 502. In various embodiments, the one or more walls 506a, 506b, 506c, 506d may be substantially perpendicular to the surfaces 302, 504 of the planar element 502. In various embodiments, the one or more walls 506a, 506b, 506c, 506d may abut or be joined or connected to the interior surface 504 of the planar element 502. In various embodiments, the planar element 502 and the one or more walls 506a, 506b, 506c, 506d may be comprised of a single component (e.g., molded plastic). In various embodiments, the planar element 502 and the one or more walls 506a, 506b, 506c, 506d may include two or more joined components.

In various embodiments, a first portion 102 of a housing 101 may include one or more corner elements 520a, 520b, 520c, 520d extending away from an interior surface 504 a planar element 502 of the housing 101. In various embodiments, the corner elements 520a, 520b, 520c, 520d may be positioned substantially along at least a portion of a perimeter 503 of the planar surface 502. In various embodiments, the one or more corner elements 520a, 520b, 520c, 520d may be substantially perpendicular to the surfaces 302, 504 of the planar element 502. In various embodiments, the one or more corner elements 520a, 520b, 520c, 520d may abut or be joined or connected to the interior surface 504 of the planar element 502. In various embodiments, the planar element 502 and the one or more corner elements 520a, 520b, 520c, 520d may be formed by a single component (e.g., molded plastic). In various embodiments, the planar element 502 and the one or more corner elements 520a, 520b, 520c, 520d may include two or more joined components.

In various embodiments, one or more walls 506a, 506b, 506c, 506d may be connected or joined to one or more corner elements 520a, 520b, 520c, 520d adjacent to or on a perimeter 503 of the planar element 502 to completely encircle the planar element 502. For example, in many embodiments, a first wall 506a may be connected or joined to a second wall 506b by a corner element 520a, the second wall 506b may be connected or joined to a third wall 506c by a corner element 520b, the third wall 506c may be connected or joined to a fourth wall 506d by a third corner element 520c, and the fourth wall 506d may be connected or joined to the first wall 506a by a fourth corner element 520d.

In other embodiments, one or more walls 506a, 506b, 506c, 506d may be connected or joined to one or more corner elements 520a, 520b, 520c, 520d along a perimeter 503 of the planar element 502 to partially encircle the planar element 502.

In various embodiments, one or more walls 506a, 506b, 506c, 506d and one or more corner elements 520a, 520b, 520c, 520d may share an exterior surface 530 that may be joined or connected to a shared interior surface 532 by a shared top surface 536. In some embodiments, a shared seam 514 may extend along a perimeter 503 of a planar element 502 and join or connect an interior surface 504 of the planar region 502 to an interior surface 504 of the one or more walls 506a, 506b, 506c, 506d and corner elements 520a, 520b, 520c, 520d. In some embodiments, a shared tapered region 515 may extend along a perimeter 503 of a planar element 502 and join or connect an interior surface 504 of the planar region 502 to an interior surface 504 of the one or more walls 506a, 506b, 506c, 506d and corner elements 520a, 520b, 520c, 520d. In some embodiments, a seam 514 may include a portion that connects a tapered region 515 to a wall 504 of the planar region 502 to an interior surface 504 of the one or more walls 506a or corner element 520a, 520b, 520c, 520d.

In various embodiments, one or more walls 506a, 506b, 506c, 506d and one or more corner elements 520a, 520b, 520c, 520d of a perimeter wall 560 may include a thick region 508 connected or joined to a thin region 512 by a lip 510 or a seam. In various embodiments, an interior surface 504 of the perimeter wall 560 may include an interior surface 532 of the thick region 508 and an interior surface 534 of the thin region 512 connected or joined by the lip 510 or seam.

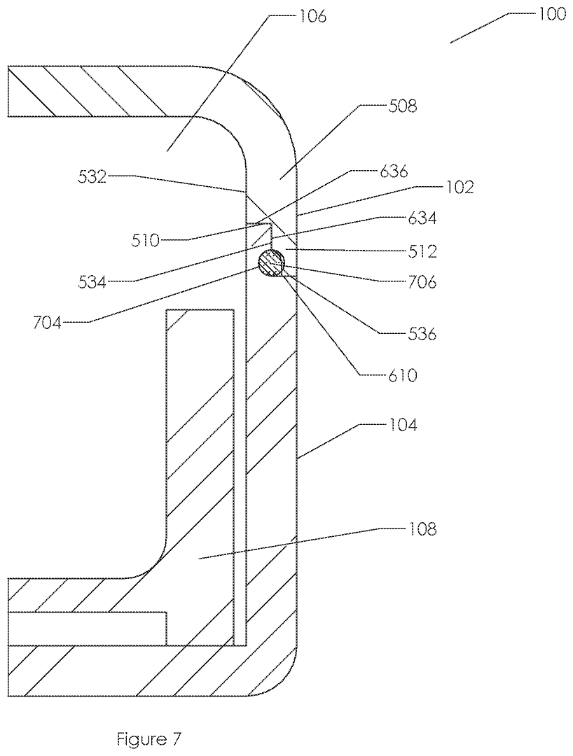

In various embodiments, a top surface 536 may be substantially planar. In other embodiments, a top surface 536 may comprise a groove for receiving a gasket or an O-ring. In various embodiments, the groove (See, e.g., FIG. 7) may extend contiguously across all of the one or more walls 506a, 506b, 506c, 506d and one or more corner elements 520a, 520b, 520c, 520d. In other embodiments, a groove may be non-contiguous. In various embodiments, the groove may be in physical proximity, abut, or be filled or partially filled by an O-ring or a gasket.

In various embodiments, one or more corner elements 520a, 520b, 520c, 520d may be substantially rounded. In some embodiments, one or more corner elements 520a, 520b, 520c, 520d may be formed by an edge of a wall 506a, 506b, 506c, 506d being connected or joined to an edge of an adjacent wall 506a, 506b, 506c, 506d.

In various embodiments, all or a portion of a first portion 102 may be formed from a rigid or substantially rigid material. In alternative embodiments, all or a portion of the first portion 102 may be formed from a flexible material. In many embodiments, all or a portion of the first portion 102 may be formed from one or more pieces of molded plastic. In alternative and additional embodiments, all or a portion of the first portion 102 may include other materials (e.g., metal, glass, plastic etc.). In various embodiments, one or more materials may be selected for constructing the systems and apparatuses described herein to be able to maintain structural integrity (e.g., resisting puncture) when coming into contact with medical equipment (e.g., sharps such as needles, razors, and/or cutting devices). In various embodiments, all or a portion of the first portion 102 may include a transparent material. For example, at least a portion or all of a planar element 502 may be transparent so that a user (e.g., a medical practitioner) can observe the contents within an internal cavity 106. In various embodiments, the planar element 502 may include a window. In various embodiments, the planar element 502 may be a window. In various embodiments, all or a portion of the first portion 102 may include an opaque material (e.g., plastic and a color additive).

Now referring to FIG. 6, a perspective view of a second portion 104 of a housing 101 for an apparatus for storing and transporting sharp or hazardous objects is illustrated in accordance with various embodiments. In various embodiments, the second portion 104 may act as a chamber and a first portion 102 may act as a lid (e.g., See FIG. 5).

In various embodiments, a second portion 104 of a housing 101 may include a planar element 602 having an interior surface 604 and an opposing exterior surface 402 (e.g., See FIG. 4). In various embodiments, the planar element 602 may include a perimeter 603 encircling the planar element 602.

In various embodiment, a perimeter wall 660 may extend away from an interior surface 604 of a planar element 602. In various embodiments, the perimeter wall 660 may be positioned adjacent to and encircle or partially encircle a perimeter 603 of the planar surface 602. In various embodiments, the perimeter wall 660 may include one or more walls 606a, 606b, 606c, 606d and one or more corner elements 620a, 620b, 620c, 620d.

In various embodiments, a second portion 104 of a housing 101 may include one or more walls 606a, 606b, 606c, 606d extending away from an interior surface 604 a planar element 602 of the housing 601. In various embodiments, the walls 606a, 606b, 606c, 606d may be positioned substantially adjacent to or on a perimeter 603 of the planar surface 602. In various embodiments, the one or more walls 606a, 606b, 606c, 606d may be substantially perpendicular to the surfaces 402, 604 of the planar element 602. In various embodiments, the one or more walls 606a, 606b, 606c, 606d may abut or be joined or connected to the interior surface 604 of the planar element 602. In various embodiments, the planar element 602 and the one or more walls 606a, 606b, 606c, 606d may be formed from a single piece of material (e.g., molded plastic). In various embodiments, the planar element 602 and the one or more walls 606a, 606b, 606c, 606d may include two or more joined components or pieces of material.

In various embodiments, a second portion 104 of a housing 101 may include one or more corner elements 620a, 620b, 620c, 620d extending away from an interior surface 604 a planar element 602 of the housing 101. In various embodiments, the corner elements 620a, 620b, 620c, 620d may be positioned adjacent to or on at least a portion of a perimeter 603 of the planar surface 602. In various embodiments, the one or more corner elements 620a, 620b, 620c, 620d may be substantially perpendicular to the surfaces 402, 604 of the planar element 602. In various embodiments, the one or more corner elements 620a, 620b, 620c, 620d may abut or be joined or connected to the interior surface 604 of the planar element 602. In various embodiments, the planar element 602 and the one or more corner elements 620a, 620b, 620c, 620d may be formed by a single component (e.g., molded plastic). In various embodiments, the planar element 602 and the one or more corner elements 620a, 620b, 620c, 620d may include two or more joined components.

In various embodiments, one or more walls 606a, 606b, 606c, 606d may be connected or joined to one or more corner elements 620a, 620b, 620c, 620d along a perimeter 603 of the planar element 602 to completely encircle the planar element 602. For example, in many embodiments, a first wall 606a may be connected or joined to a second wall 606b by a corner element 620a, the second wall 606b may be connected or joined to a third wall 606c by a corner element 620b, the third wall 606c may be connected or joined to a fourth wall 606d by a third corner element 620c, and the fourth wall 606d may be connected or joined to the first wall 606a by a fourth corner element 620d.

In other embodiments, one or more walls 606a, 606b, 606c, 606d may be connected or joined to one or more corner elements 620a, 620b, 620c, 620d along a perimeter 603 of the planar element 602 to partially encircle the planar element 602.

In various embodiments, one or more walls 606a, 606b, 606c, 606d and one or more corner elements 620a, 620b, 620c, 620d may share an exterior surface 630 that may be joined or connected to a shared an interior surface 631 by a shared top surface 636. In some embodiments, a shared seam 614 may extend along a perimeter 603 of a planar element 602 and join or connect an interior surface 604 of the planar region 602 to an interior surface 604 of the one or more walls 606a, 606b, 606c, 606d and corner elements 620a, 620b, 620c, 620d.

In various embodiments, one or more walls 606a, 606b, 606c, 606d and one or more corner elements 620a, 620b, 620c, 620d of a perimeter wall 660 may include a thick region 608 connected or joined to a thin region 612 by a lip 610 or a seam. In various embodiments, an exterior surface 630 of the perimeter wall 660 may include an exterior surface 632 of the thick region 608 and an exterior surface 634 of the thin region 612 connected or joined by the lip 610 or seam.

In various embodiments, a top surface 636 may be substantially planar. In other embodiments, a top surface 636 may comprise a groove for receiving a gasket or O-ring. In various embodiments, the groove may extend contiguously across all of the one or more walls 606a, 606b, 606c, 606d and one or more corner elements 620a, 620b, 620c, 620d. In other embodiments, a groove may be non-contiguous. In various embodiments, the groove may be in physical proximity, abut, or be filled or partially filled by an O-ring or a gasket.

In various embodiments, one or more corner elements 620a, 620b, 620c, 620d may be substantially rounded. In some embodiments, one or more corner elements 620a, 620b, 620c, 620d may be formed by an edge of a wall walls 606a, 606b, 606c, 606d being connected or joined to an edge of an adjacent wall walls 606a, 606b, 606c, 606d.

In various embodiments, all or a portion of a second portion 104 may be formed from a rigid or substantially rigid material. In alternative embodiments, all or a portion of the second portion 104 may be formed from a flexible material. In many embodiments, all or a portion of the second portion 104 may be formed from one or more pieces of molded plastic. In alternative and additional embodiments, all or a portion of the second portion 104 may include other materials (e.g., metal, glass, etc.). In various embodiments, one or more materials may be selected for constructing the systems and apparatuses described herein to be able to maintain structural integrity (e.g., resisting puncture) when coming into contact with medical equipment (e.g., sharps such as needles, razors, and/or cutting devices). In various embodiments, all or a portion of the second portion 104 may include a transparent material. For example, at least a portion or all of a planar element 602 may be transparent so that a user (e.g., a medical practitioner) can observe the contents within an internal cavity 106. In various embodiments, the planar element 602 may include a window. In various embodiments, the planar element 602 may be a window. In various embodiments, all or a portion of the second portion 104 may include an opaque material (e.g., plastic and a color additive).

In various embodiments, a first portion 102 of a housing can be placed into physical contact with a second portion 104 of the housing 101 by a medical practitioner to seal the contents (e.g., medical equipment and/or medical waste) into an internal cavity 106 of the apparatus for storing and transporting sharp or hazardous objects in accordance with various embodiments 100. In various embodiments, the first portion 102 and the second portion 104 may be formed from separate components and may be fitted together by the medical practitioner. In various embodiments, the first portion 102 and the second portion 104 may be connected by a connector 103 and the connector 103 may be actuated to move the first portion 102 into physical contact with the second portion 104. In various embodiments, the seal may be leak-proof. In various embodiments, the seal may be leak-resistant.

In various embodiments, an apparatus for storing and transporting sharp or hazardous objects 100 in a closed position may seal an internal cavity 106. In various embodiments, the internal cavity 106 be leak-proof. In various embodiments, the internal cavity 106 may be leak-resistant. In various embodiments, the internal cavity 106 may be sealed by abutting one or more of the surfaces of a first portion 102 against one or more of the surfaces of the second portion 104 (e.g., see FIG. 7). A top surface 536 of a perimeter wall 560 (e.g., a shared top surface 536 of one or more walls 506a, 506b, 506c, 506d and one or more corner elements 520a, 520b, 520c, 520d) of the first portion 102 may abut or be in physical proximity to a lip 610 of the second portion 104 while the apparatus for storing and transporting sharp or hazardous objects 100 is in the closed position, thereby forming a seal or partial seal in accordance with various embodiments. A top surface 636 of a perimeter wall 660 (e.g., a shared top surface 636 of one or more walls 606a, 606b, 606c, 606d and one or more corner elements 620a, 620b, 620c, 620d) of the second portion 104 may abut or be in physical proximity to a lip 510 of the first portion 102 while the apparatus for storing and transporting sharp or hazardous objects 100 is in the closed position (e.g., see FIGS. 3 and 4), thereby forming a seal or partial seal in accordance with various embodiments. An interior surface 534 of a thin region 512 of the perimeter wall 560 (e.g., a shared interior surface 534 of a thin region 512 of one or more walls 506a, 506b, 506c, 506d and one or more corner elements 520a, 520b, 520c, 520d) of the first portion 102 may abut or be in physical proximity to an exterior surface 634 of a thin region 612 of the perimeter wall 660 (e.g., a shared exterior surface 634 of a thin region 612 of one or more walls 606a, 606b, 606c, 606d and one or more corner elements 620a, 620b, 620c, 620d) of the second portion 104. In various embodiments, the first portion 102 and the second portion 104 may be sealed by a compressive force.

FIG. 7 is a cross-section view of a first portion 102 and a second portion 104 of a housing 101 for an apparatus for storing and transporting sharp or hazardous objects 100 in a closed position in accordance with various embodiments. In various embodiments, a sealing element 706 (e.g., a gasket or an O-ring) can insert into a groove 704 to create a seal between the first portion 102 of the housing 101 and the second portion 104 of the housing 101. The seal can prevent an internal cavity 106 from leaking contents out into a surrounding environment. In various embodiments, a groove 704 may extend along a perimeter wall 560 of a first portion 102. In various embodiments, a groove 704 may extend along a perimeter wall 660 of a second portion 104. In various embodiments, a groove may extend along a top surface 536 of the perimeter wall 560 of the first portion 102. In various embodiments, a groove 704 may extend along a surface (e.g., a top surface, a lip, a surface of a thin region) of a first portion 102 or a second portion 104. In various embodiments, a groove 704 may extend along a lip 510 of the first portion 102. In various embodiments, a groove 704 may extend along a lip of the second portion 104. In various embodiments, a groove may extend along an interior surface 534 of a thin region 512 of the first portion 102. In various embodiments, a groove 704 may extend along a surface 702 of a thin region of the second portion 700. In some embodiments, a surface 536, 636, 510, 610, 534, 634 may include one or more grooves 704 to accommodate one or more sealing elements 706 (e.g., a gasket or O-ring). Embodiments including multiple sealing elements 706 (e.g., gaskets or O-rings) may increase leak resistance.

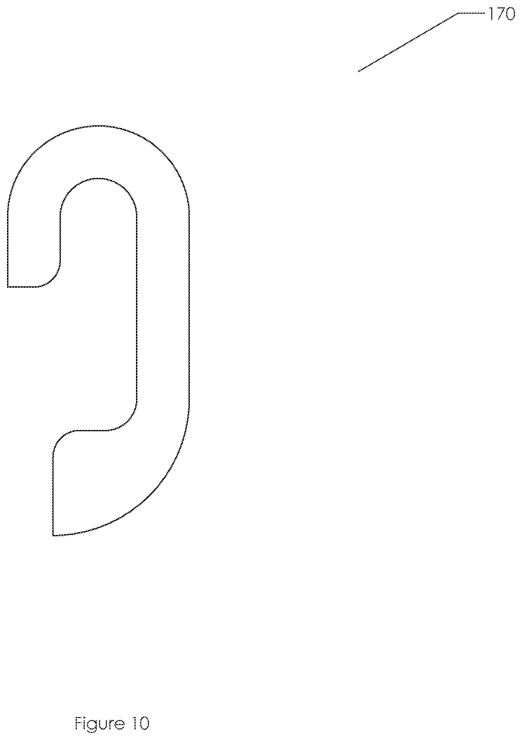

FIG. 10 is an illustration of a removeable element 170 that can also been seen in FIG. 1 in accordance with various embodiments. In various embodiments, the removeable element 170 may be positioned along a wall of a second portion. In various embodiments, the removeable element 170 may interact with a removeable sidewall 181 (e.g., see FIGS. 1-5) positioned on the first portion of the housing to remove a securing element from engaging. More specifically, a removeable element 170 may be shaped roughly like a “c” or a “u” where a first portion wraps over a sidewall to interact with an interior surface of a second portion (e.g., a sidewall) and a second portion abuts an external surface of a second portion (e.g., an external surface of a sidewall).

In various embodiments, a removeable element 170 may interact with a removeable sidewall 181. In various embodiments, once the removeable sidewall 181 is removed there are no surfaces for the removeable element 170 to interact which may allow for one or more securing elements (e.g., first and second elements) to interact and seal a first portion of a housing to a second portion of a housing.

In some embodiments, a removeable element may be positioned between a first element and a second element of a securing element in accordance with various embodiments. In various embodiments, the first element may be positioned on an exterior surface of the first portion. In other and additional embodiments, the first element may be positioned on an interior surface of the first portion. In various embodiments, the second element may be positioned on an exterior surface of the second portion. In other and additional embodiments, the second element may be positioned on an interior surface of the second portion. In various embodiments, the removeable element may prevent the first element from interacting (e.g., latching to) the second element. In some embodiments, the securing element may be inaccessible to a user and once the removeable element is removed and the first element interlocks with the second element the first portion and the second portion be locked together. In some embodiments, a portion of the removeable element may be exposed to an exterior of a housing 101 of the first portion 102 and the second portion 104 while the securing element may be located in an internal cavity 106 (or on an interior surface 532, 534, 632, 634) of the housing 101. In such embodiments, a user may close the housing 101 (e.g., connecting the first portion 102 to the second portion 104), remove the removeable element, and permanently secure the first portion 102 to the second portion 104 using the securing element. In various embodiments, the first element may include an angled surface and the second element may include an angled surface. As the angled surfaces slide against one another (e.g., as the first portion 102 moves toward the second portion 104) a flexible portion of the first element and/or a flexible portion of the second element may deform and once the first portion 102 abuts the second portion 104 one or both of the flexible portions may snap back into its original position and a locking surface of the first element and a locking surface of the second element may abut one another, thereby, sealing the first portion 102 to the second portion 104 to enclose an internal cavity 106 of a housing 101 of an apparatus. In such embodiments, a user may not have the ability to access a cavity containing hazardous material, medical waste, or sharps. In various embodiments, the apparatus may be disposed of by transportation to a collection site, a needle drop-off site, a gas station or market drop-off site (e.g., a gas stations or airport), an ambulance drop-off site, and then be later destroyed elsewhere or at the site.

In various embodiments, a removeable sidewall 181 (see FIGS. 1-5) may be joined to one or more of the first and second elements on the first portion of the housing. In various embodiments, the removeable sidewall 181 may prevent the locking surfaces from interacting with each other to secure a first portion of the housing to a second portion of the housing. In various embodiments, the removeable sidewall 181 may press against part of the removeable element 170, thereby, pushing the elements and/or locking surfaces away from each other. In various embodiments, the removeable sidewall 181 may be joined to one or more of the elements by a perforated portion so that a user may easily remove the removeable sidewall 181. In various embodiments, when the removeable sidewall 181 has been removed the locking surfaces may interact to permanently close the housing. Once permanently closed, any sharp objects contained within an interior of the housing will be safely out of reach along with any contaminants.

Referring to FIG. 3, a housing 101 for an apparatus for storing and transporting sharp or hazardous objects 100 may include a securing element 304a, 304b. In various embodiments, the securing element 304a, 304b may ensure a first portion 102 of a housing 101 stays connected to a second portion 104 of the housing 101. In various embodiments, the securing element 304a, 304b may comprise a first element 306a, 306b and a second element 308a, 308b. In various embodiments, the first portion 102 and the first element 306a, 306b may be molded from a single piece of material (e.g., molded plastic). In some embodiments, the first portion 102 and the first element 306a, 306b may be molded from two or more pieces of material (e.g., molded plastic). In some embodiments, the first element 306a, 306b may be connected, joined or be affixed to the first portion 102. In various embodiments, the second portion 104 and the second element 308a, 308b may be molded from a single piece of material (e.g., molded plastic). In some embodiments, the second portion 104 and the second element 308a, 308b may be molded from two or more pieces of material (e.g., molded plastic). In various embodiments, the second element 308a, 308b may connect or be affixed to the second portion 104. In various embodiments, the first element 306a, 306b of the first portion 102 may interact with the second element 308a, 308b of the second portion 104 while the housing 101 is in the closed position. In various embodiments, the first element 306a, 306b can be actuated to interact with the second element 308a, 308b (e.g., a hook catching a clasp). In various embodiments, the second element 308a, 308b may be actuated to interact with the first element 306a, 306b (e.g., a hook and a clasp). In various embodiments, the interaction may be physical. For example, the first element 306a, 306b of the first portion 102 and the second element 308a, 308b of the second portion 104 act to apply a force holding the first portion 102 against the second portion 104. In various embodiments, the force applies the compressive forces described herein. In various embodiments, the force may be applied to one or more surface 536, 534, 510 of the first portion 102 against one or more surfaces 636, 634, 610 of the second portion 104 to create the seal as described herein. In some embodiment, the force may act to compress a sealing element (e.g., a gasket or an O-ring) to create the seal as described herein.

In various embodiments, a first element 306a, 306b may be positioned on an exterior surface 530 of a first portion 102. In various embodiments, a second element 308a, 308b may be positioned on an exterior surface 630 of a second portion 104. In various embodiments, a first element 306a, 306b may be positioned on the exterior surface of a wall 506a of the first portion 102. In some embodiments, the first element 306a, 306b may be positioned on the exterior surface of a wall 506b of the first portion 102. In some embodiments, the first element 306a, 306b may be positioned on the exterior surface of a wall 506c of the first portion 102. In some embodiments, the first element 306a, 306b may be positioned on the exterior surface of a wall 506d of the first portion 102. In various embodiments, the second element 308a, 308b may be positioned on the exterior surface of a wall 606a of the second portion 104. In some embodiments, the second element 308a, 308b may be positioned on the exterior surface of a wall 606b of the second portion 104. In some embodiments, the second element 308a, 308b may be positioned on the exterior surface of a wall 606c of the second portion 104. In some embodiments, the second element 308a, 308b may be positioned on the exterior surface of a wall 606d of the first portion 104.

In various embodiments, a securing element 304a, 304b may include a latch system. Non-limiting examples of latch systems may include cam latches, compression latches, slam latches, draw latches, and sliding latches. In various embodiments, the latch system may include a snap latch. For example, a first element 306a, 306b may include a hook and a second element 308a, 308b may include an opposing hook affixed to or part of a flexible portion. In various embodiment, the hook may engage the opposing hook as a first portion 102 moves toward a second portion 104, thereby causing the flexible portion to flex which displaces the opposing hook until the close position is reached, thereby causing the flexible portion to snap the opposing hook into an interlocking configuration with the first hook.

In various embodiments, an apparatus for storing and transporting sharp or hazardous objects 100 may include a securing element 304a, 304b having a locking mechanism preventing a closed configuration (See FIGS. 3 and 4) from opening (See FIGS. 1 and 2).

In various embodiments, a securing element 304a, 304b may be positioned on an interior surface 532, 534, 631 of a perimeter wall 560, 660 of a housing 101. In various embodiments, a first element 306a, 306b of a first portion 102 may be positioned on an interior wall 532, 534 and a second element 308a, 308b of a second portion 104 may be positioned on an interior surface 631. In some embodiments, the securing element 304a, 304b may not be accessible once the first portion 102 connects or abuts the second portion 104 of the housing 101. As such, medical waste and/or medical equipment sealed within the internal cavity 106 may no longer pose a threat to a medical practitioner. In some embodiments, a removeable element (e.g., a tab) may physical obstruct may prevent a first element 306a, 306b from physically interacting with a second element 308a, 308b until a medical practitioner has removed the obstruction.

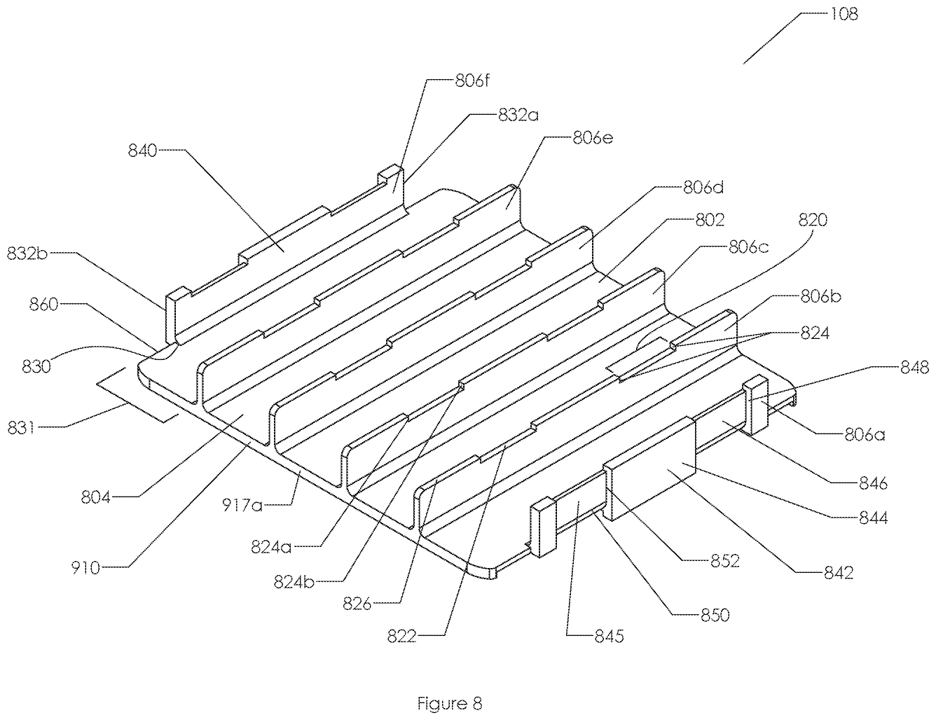

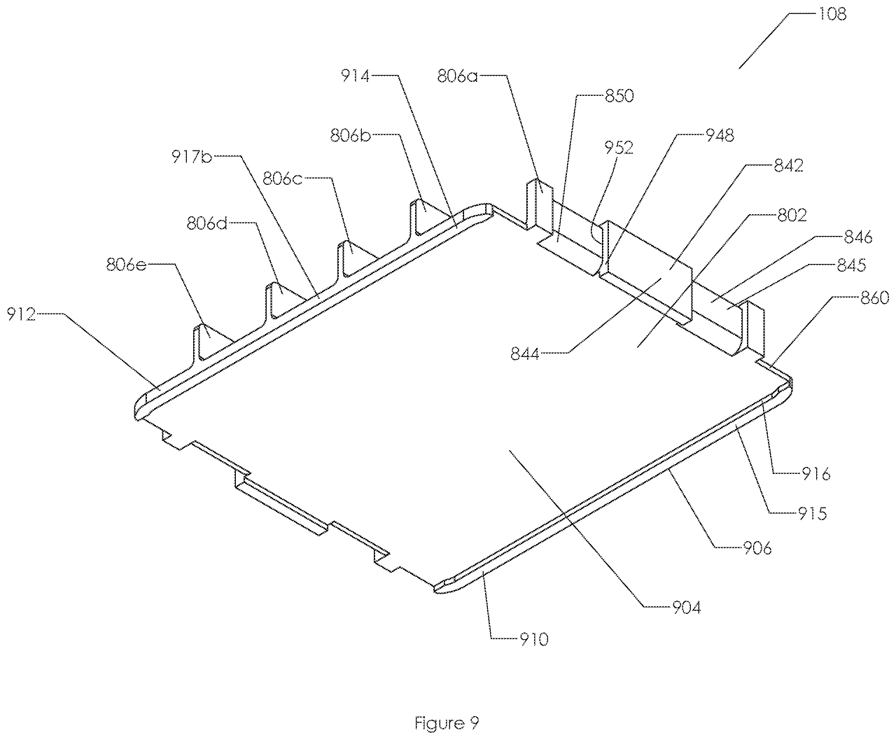

FIG. 8 is a perspective view of a structural element 108 of an apparatus for storing and transporting sharp or hazardous objects 100 in accordance with various embodiments and FIG. 9 is an opposing perspective view of the structure element 108. Once a user or medical practitioner has used a piece of medical equipment and/or medical waste it can be deposited into an internal cavity 106 of a housing 101 in accordance with various embodiments. Non-limiting uses for the structural element 108 may include dividing or separating contents (e.g., medical equipment and/or medical waste) and restricting movement of the contents. In various embodiments, using both the structural element 108 and a restrictive element 110 in combination may secure and restrict movement of a piece of medical equipment (e.g., a contaminated needle). The restrictive element 110 may comprise a strap or an elastic band (see FIG. 1).

In various embodiments, a structural element 108 may comprise a floor 802 and one or more walls 806a, 806b, 806c, 806d, 806e, 806f. In some embodiments, the floor 802 may be indistinguishable from a planar element 502 of a second portion 104 of a housing 101. For example, the planar element 502 and the floor 802 may comprise one contiguous, substantially flat surface and/or the second portion 104 and the structural element 108 may be a single component (e.g., resulting from a same injection mold) in accordance with some embodiments.

In other and various embodiments, a structural element 108 include one or more separate components from a second portion 104 and later be inserted and connected or joined to the second portion 104.

In various embodiments, each of one or more walls 806a, 806b, 806c, 806d, 806e, 806f include at least one recess 820 for receiving a restrictive element 110. In various embodiments, a wall 806a, 806b, 806c, 806d, 806e, 806f may include a bottom surface 830 and two outward surfaces 832a, 832b extending away from the bottom surface 830 to meet an upper surface 826. In various embodiments, a recess 820 may be formed by two side surfaces 824a, 824b extending downward from the upper surface 826 to a lower surface 822. In many embodiments, the recess 820 may be rectangular in shape. In some embodiments, the recess 820 may include a single rounded surface (e.g., appear as a “dip” or semi-circle). In various embodiments, a recess 820 shape may be selected to accommodate a shape of a restrictive element 110. In various embodiments, a wall 806a, 806b, 806c, 806d, 806e, 806f having two or more recesses 820 may take the appearance of “teeth” (e.g., See FIG. 8).

In various embodiments, a structural element 108 may comprise a first outer wall 806a and a second outer wall 806f including a conformation that may be different from interior walls 806b, 806c, 806d, 806e. In various embodiments, the outer walls 806a, 806f may comprise a thick region 844 joined or connected to a thin region 845. In various embodiments, the outer walls 806a, 806f may include a recess 820 may be formed by two side surfaces 824a, 824b extending downward from the upper surface 826 to a lower surface 822. In various embodiments, the lower surface 822 may form a right angle to meet a restrictive element receiving surface 846. In various embodiments, a curved surface 850 may connect or join the restrictive element receiving surface 846 to the opposing second surface 904. In various embodiments, the curved surface 850 may provide a smooth surface for contacting the restrictive element 110, thereby, preventing damage to the restrictive element 110. In various embodiments, the restrictive element 110 may be in physical contact with one or more recesses 820, at least one restrictive element receiving surface 846, and a second surface 904 of the 802 floor.

In various embodiments, a thin region 845 may comprise a first sidewall 852 joined or connected to a first sidewall 848 of a thick region 844. In various embodiments, a thin region 845 may comprise a second sidewall 952 joined or connected to a second sidewall 948 of a thick region 844.

In various embodiments, a floor 802 of a structural element 108 may include a planar surface having a first surface 804 and an opposing second surface 904. In various embodiments, the structural element 108 may comprise two substantially parallel rails 910, 912 connected or joined to the second surface 904 of the floor 802. In various embodiments, the rails 910, 912 may extend away and perpendicular from the second surface 904 of the floor 802.

In various embodiments, a first rail 910 may comprise an interior surface 916, an opposing exterior surface 917a, joined by a top surface 915. In various embodiments, a bottom surface (not shown) of the first rail 910 may be joined or connected to the second surface 904 of the floor 802. In various embodiments, the top surface 915 may be joined or connected to an interior surface 604 of a planar element 602 of a second portion 104 (see FIG. 6).

In various embodiments, a second rail 912 may comprise an interior surface (not shown), an opposing exterior surface 917b, joined by a top surface 914. In various embodiments, a bottom surface (not shown) of the second rail 912 may be joined or connected to the second surface 904 of the floor 802. In various embodiments, the top surface 914 may be joined or connected to an interior surface 604 of a planar element 602 of a second portion 104 (see FIG. 6).

In various embodiments, a perimeter surface 860 may encircle or partially encircle the floor 802 of the structural element 108 and may connect or join the first surface 804 to the opposing second surface 904. In various embodiments, the perimeter surface 860 may comprise an exterior surface 917a of a first rail 910. In various embodiments, the perimeter surface 860 may comprise an exterior surface 917b of a second rail 912. In various embodiments, at least a portion of the perimeter surface 860 may comprise outer surface 842 of a thick region 844. In various embodiments, at least a portion of the perimeter surface 860 may comprise a restrictive element receiving surface 846 of a thin region 845. In various embodiments, at least a portion of the perimeter surface 860 may comprise at least a portion of a curved region 850 of a thin region 845.

In various embodiments, a floor 802 of a structural element 108 and one or more walls 806a, 806b, 806c, 806d, 806e, 806f of the structural element 108 may be formed from a single component (e.g., molded plastic). In other embodiments, the one or more walls 806a, 806b, 806c, 806d, 806e, 806f may be formed separately (e.g., two or more pieces of molded plastic) and then be connected or joined to the floor 802. In various embodiments, a bottom surface 830 of each of the one or more walls 806a, 806b, 806c, 806d, 806e, 806f may be connected or joined to a first surface 804 of the floor 802.

In various embodiments, a structural element 108 may be connected or joined to a planar element 602 of a second portion 104 of a housing 101. For example, a floor 802 of the structural element 108 may be connected or joined to the planar element 602. In various embodiments, the structural element 108 and the second portion 104 may detachably couple such that different structural elements 108 may be swapped out of the second portion 104.

In various embodiments, one or more attachment features may be used to secure the floor 802 of the structural element 108 to the planar element 602 of the second portion 104. In some embodiments, the one or more attachment features may be configured to interact with an opposing attachment feature of the second portion 104. Non-limiting examples of attachment features and opposing attachment features include tacks, staples, holes, pins, or any other device(s) known to couple two objects to one another.

In other embodiments, a second surface 904 of a structural element 108 may be joined to an interior surface 604 of a planar element 602. In some embodiments, joining may include applying an adhesive to the surfaces 904, 604 and placing them in physical contact with one another. In other embodiments, joining may include welding along a perimeter 906 of a floor 802 of the structural element 108 to the interior surface 604 of the planar element 602.

As described herein, in various embodiments one or more rails 910, 912 may be joined or connected to an interior surface 604 of a planar element 602 of a second portion 104 of a housing 101. In these embodiments, a space between a second surface 904 of a structural element 108 and the interior surface 604 of the second portion 104 may accommodate at least a portion of a restrictive element 110. For example, in some embodiments, the restrictive element 110 may comprise a contiguous loop extending all the way around the structural element 108 (e.g., contacting one or more recesses 820, one or more restrictive element receiving surfaces 846, and a second surface 904 of the structural element 108). In many embodiments, the restrictive element 110 may comprise a rubber band.

Referring to FIGS. 1, 8, and 9, a restrictive element 110 in combination with a structural element 108 to secure may be used to restrict movement of a piece of medical equipment (e.g., a contaminated needle). For example, a restrictive element 110 may secure and restrict movement of a piece of medical equipment in a channel 831 of the structural element 108 by acting as a physical barrier and/or applying a force against the piece of medical equipment, thereby, pressing it against one or more of the surfaces of the channel 831. In some embodiments, the structural element 108 may include one channel 831. In various embodiments, the structural element 108 include at least two channels 831. In various embodiments, the at least two channels 831 may be similar in size and/or shape. In other embodiments, at least some of the at least two channels 831 may vary in size and/or shape. For example, in embodiments where the housing 101 resembles a clamshell, the at least two channels 831 may vary in size and shape. In various embodiments, the first portion 102 and/or the second portion 104 may include a curved surface. In various embodiments, an exterior surface 302 of the first portion may include texture or a non-slip element. In various embodiments, an exterior surface 402 of the first portion may include texture or a non-slip element. The texture or non-slip element may include a coarse material or molding, in accordance with some embodiments. The texture or non-slip element may include uneven surfaces.

Channels 831 may be sized to accommodate diabetic toadstool needles, EpiPens®, 2-pam injectors, atropine injectors, autoinjectors, antidote needles, Lovenox® prefilled syringes, insulin syringes, intermuscular syringes, intramuscular syringes, syringes designed to inject subcutaneously. Botox syringes, lidocaine syringes, sedative syringes, loose needles and/or associated with syringes of varying sizes (e.g., 10 gauge-34 gauge), syringes designed for NARCAN® injections, or any other needle (injection, intravenous, etc.) and/or syringe type used in the medical fields.

In various embodiments, a restrictive element 110 may extend across one or more recesses 820 of one or more walls 806a, 806b, 806c, 806d, 806e, 806f of a structural element 108. In various embodiments, a first attachment of the restrictive element 110 may connect or join to first position of an interior surface 631 of a wall 606a, 606b, 606c, 606d or corner 620a, 620b, 620c, 620d of a second portion 104 and a second attachment of the restrictive element 110 may connect or join to a second position of an interior surface 631 of a wall 606a, 606b, 606c, 606d or corner 620a, 620b, 620c, 620d of the second portion 104. In other embodiments, a first attachment of the restrictive element 110 may connect or join to a first wall of the structural element 108 and a second attachment of the restrictive element 110 may connect or join to a second wall of the structural element 108.

Non-limiting examples of attachments may include adhesive, tacks, pins, staples, screws, or any other known device capable of connecting or joining two objects.

In various embodiments, a restrictive element 110 may include an elongated elastic material (e.g., a rubber band).

In some embodiments, a restrictive element 110 may include a rigid material such as plastic or metal. In such embodiments, at least one of the attachments may include a hinge such that the restrictive element 110 can swing into place once used medical equipment has been placed into a channel 831 of a structural element 108.

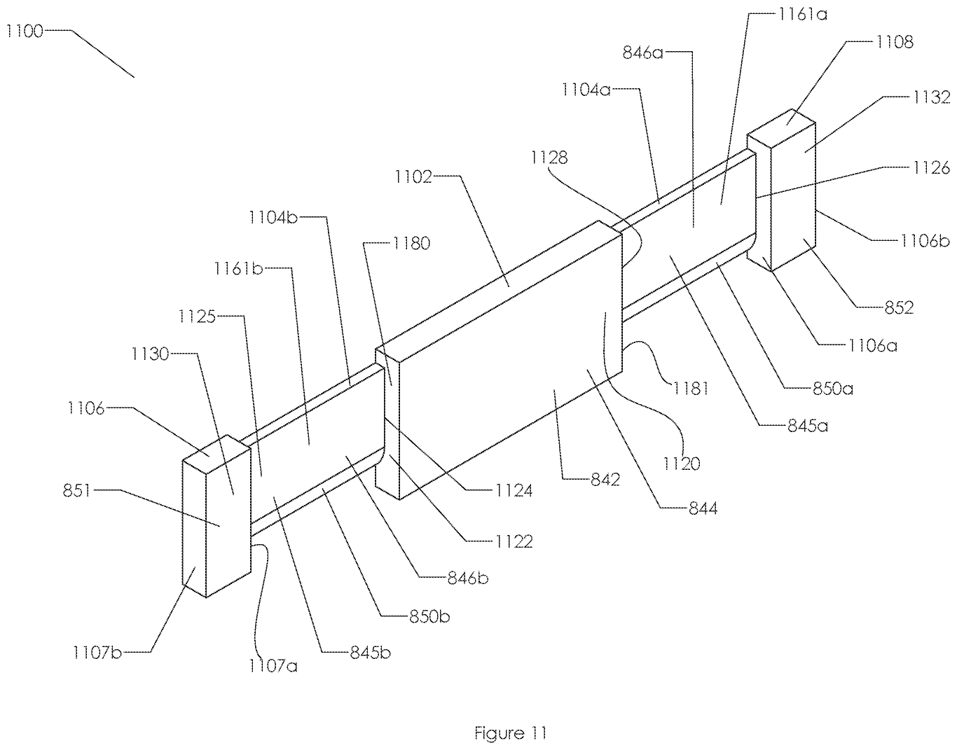

FIG. 11 is an illustration of a wall 1100 of a structural element 108 in accordance with various embodiments. In various embodiments, the structural element 108 comprises two walls. In various embodiments, the structural element 108 comprises at least two walls. In various embodiments, the structural element 108 comprises more than two walls.

In various embodiments, a wall 1100 may comprise at least one thick region 844 and at least one thin region 845a, 845b. In various embodiments, the wall 1100 may comprise at least one end cap 1130, 1132. In various embodiments, a cross-sectional area of the thick region 844 may be greater than a cross sectional area of the thin region 845a, 845b. Such a configuration may be ideally suited for eliminating or reducing lateral movement of a restrictive element 110. For example, a restrictive element 110 may be in physical contact with one or more of the surfaces 1104a, 1104b, 1161a, 1161b, 850a, 850b (as described herein) of the thin region 845a, 845b, in accordance with various embodiments. When the cross-sectional area of the thick region 844 is greater than the cross-sectional area of the thin region 845a, 845b, side surfaces 1180, 1181 and side surfaces 1107, 1106 on the end caps 1130, 1132 may be exposed to form a lip that may assist in restricting lateral movement of the restrictive element 110.

In various embodiments, a thick region 844 may include a first end 1120 and an opposing second end 1122. In various embodiments, the thick region may include a height and a width. In various embodiments, a first thin region 845a may be joined to the thick region 844. In various embodiments, a second thin region 845b may be joined to the thick region 844. In various embodiments, the first thin region 845a and the second thin region 845b may be joined to the thick region 844 at opposing locations.

In various embodiments, the first thin region 845a may include a first end 1128 and an opposing second end 1126. In various embodiments, the first thin region 845a may include a height and a width. In various embodiments, the first end 1120 of the thick region 844 may be joined to the first end 845a of the first thin region 845a. In various embodiments, the height of the thick region 844 may be greater than the height of the first thin region 845a. In various embodiments, the width of the thick region 844 may be greater than the width of the first thin region 845b.

In various embodiments, the second thin region 845b may include a first end 1125 and a second end 1124. In various embodiments, the second thin region 845b may include a height and a width. In various embodiments, the second end 1122 of the thick region may be joined to the second end 1124 of the second thin region 845b. In various embodiments, the height of the thick region 844 may be greater than the height of the second thin region 845b. In various embodiments, the width of the thick region 844 is greater than the width of the second thin region 845b.

In various embodiments, one or more walls 1100 (e.g., a first wall and a second wall) may each comprise a first end cap 1132 may be joined to the second end 1126 of the first thin region 845a and a second end cap 1130 may be joined to the first end 1125 of the second thin region 845b. In various embodiments, a height of the first end cap 1132 and a height of the second end cap 1130 may be equal.

In various embodiments, a thin region 845a, 845b may comprise a restrictive element receiving surface 846a, 846b. In various embodiments, the restrictive element receiving surface 846a, 846b may include a top surface 1104a, 1104b of the thin region 845a, 845b. In various embodiments, the restrictive element receiving surface 846a, 846b may include an outer surface 1161a, 1161b of the thin region 845a, 845b. In various embodiments, the restrictive element receiving surface 846a, 846b may include a curved surface 850a, 850b of the thin region 845a, 845b. In various embodiments, the restrictive element receiving surface 846a, 846b may include a top surface 1104a, 1104b connected to a curved surface 850a, 850b by an outer surface 1161a, 1161b of the thin region 845a, 845b. In various embodiments, the restrictive element receiving surface 846a, 846b does not include an inner surface 840. In various embodiments, the inner surface 840 may connect the outer surface 1161a, 1161b by the top surface 1104a, 1104b. In various embodiments, the top surface 1104a, 1104b may be perpendicular to the inner surface 840 and the outer surface 1104a, 1104b. In various embodiments, the curved surface 850a, 850b may connect the inner surface 840 to the outer surface 1104a, 1104b. In various embodiments, the curved surface 850a, 850b may be located opposite to the top surface 1104a, 1104b.

In various embodiments, a wall 1100 of a structural element 108 may comprise a single and contiguous inner surface 840 spanning the first end cap 1132, the first thin region 845a, the thick region 844, the second thin region 845b, and the second end cap 1130 along a single plane.

In various embodiments, a first end cap 1132 of a wall 1100 may include two opposing side surfaces 1106a, 1106b connected by an outer surface 852 and a top surface 1108 at right angles.

In various embodiments, a second end cap 1130 of a wall 1100 may include two opposing side surfaces 1107a, 1107b connected by an outer surface 851 and a top surface 1106 at right angles.

In various embodiments, a thick region 844 of a wall 1100 may include two opposing ends 1122, 1181 connected by an outer surface 842 and a top surface 1102 at right angles.

In various embodiments, a top surface 1108 of a first end cap 1132, a top surface 1106 of a second end cap 1130, and a top surface 1102 of a thick region 844 may be aligned to a same spatial plane.

In various embodiments, an outer surface 1161a of a first end cap 1132, an outer surface 1161b of a second end cap 1130, and an outer surface 842 of a thick region 844 may be aligned to a same spatial plane.

### Exemplary Methods of Use

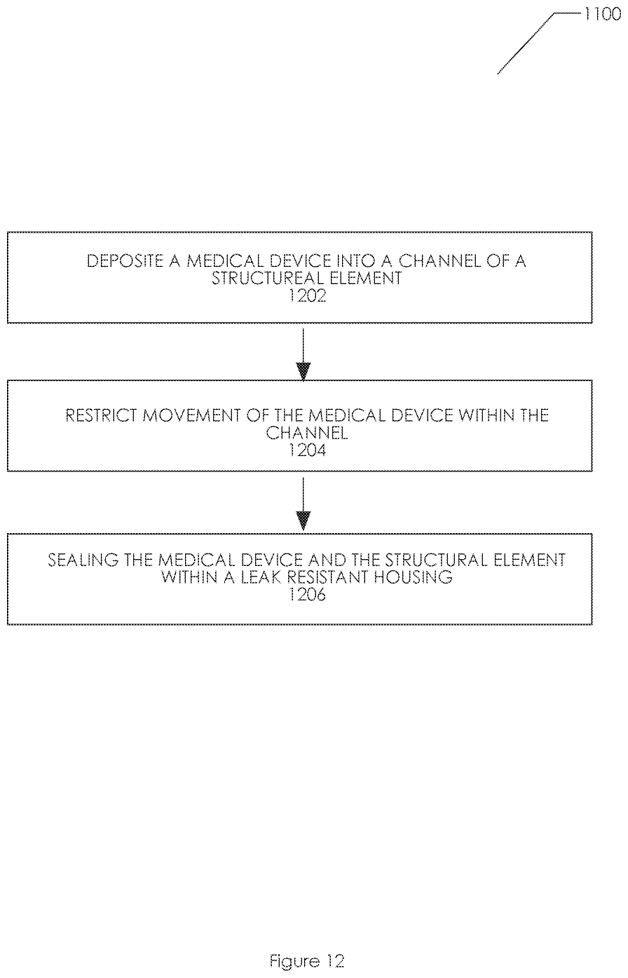

FIG. 12 is a flow chart illustrating a method of use for storing and transporting sharp or hazardous objects 1200 in accordance with various embodiments. The processes described herein describe exemplary uses of the systems and apparatuses described throughout the claims and description herein.

Step 1202 includes depositing a medical device into a channel of a structural element.

Step 1204 includes restricting movement of the medical device within the channel. In some embodiments, the step of restricting movement further comprises positioning a restrictive element over the channel. In some embodiments, the restrictive element comprises an elongated elastic portion and the method further comprises the steps of expanding the elongated elastic portion prior to the depositing step and contracting the elongated elastic portion during the restricting movement step. In additional and other embodiments, the restrictive element comprises an elongated rigid portion having a first end and a second end. The first end may comprise a hinge. The method may further include actuating the hinge to move the second end of the elongated rigid portion away from the channel of the structural element prior to the depositing step and actuating the hinge to move the second end of the elongated rigid portion over the channel of the structural element during the restricting movement step in accordance with some embodiments. In various embodiments, the structural element may include a floor, a first planar element protruding from the floor, and a second planar element protruding from the floor. In some embodiments, the first planar element comprises a first recess positioned adjacent to an upper surface. In some embodiments, the second planar element comprises a second recess positioned adjacent to an upper surface. In some embodiments, the first planar element is spaced apart from the second planar element. In some embodiments, the step of positioning the restrictive element over the channel to restrict movement of the medical device further comprises positioning a portion of the restrictive element within the first recess and another portion within the second recess.

In various embodiments, the step of restricting movement further comprises contacting the restrictive element with a restrictive element receiving surface of the first planar element and with a restrictive element receiving surface of the second planar element. In various embodiments, the step of restricting movement further comprises contacting the restrictive element with a curved surface of the first planar element and with a curved surface of the second planar element. In various embodiments, the step of restricting movement further comprises contacting the restrictive element with a second surface of the floor and the first and second protrusions are joined to an opposing first surface of the floor.

Step 1206 includes sealing the medical device and the structural element within a leak resistant housing. In some embodiments, the step of sealing the medical device within a leak resistant housing, further comprises positioning a first portion of the housing adjacent to a second portion of the housing. In some embodiments, the step of positioning the first portion of the housing adjacent to the second portion of the housing, further comprises abutting a surface of a wall of the first portion against a surface of a wall of the second portion to completely enclose an internal cavity of the leak resistant housing. In some embodiments, the step of positioning the first portion of the housing adjacent to the second portion of the housing comprises actuating a hinge, wherein a portion of the hinge is positioned on a surface of the first portion of the housing and another portion of the hinge is positioned on a surface of the second portion of the housing.

In various embodiments, the method further comprises actuating a securing element to ensure the housing stays in a closed position, wherein a first element of the securing element is positioned on the first portion and a second element of the securing element is positioned on the second portion.

In some embodiments, the method further comprises incinerating the leak resistant housing.

### EQUIVALENCE

Those skilled in the art will recognize or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

1. A system for safely storing sharp or hazardous materials, the system comprising:
a first portion of a housing;
a second portion of the housing detachably coupled to the first portion of the housing; and
a structural element positioned within an internal cavity of the second portion.

2. The system for safely storing sharp or hazardous materials of claim 1, wherein the structural element comprises:
a floor comprising a first surface and an opposing second surface;
a first wall projecting upwardly from the first surface of the floor; and
a second wall projecting upwardly from the first surface of the floor,
wherein the first surface of the floor and the two walls partially enclose a channel.

3. The system for safely storing sharp or hazardous materials of claim 2, wherein the first wall and the second wall each comprise:
a thick region including a first end, an opposing second end, a height, and a width; and
a first thin region including a first end, an opposing second end, a height, and a width,
wherein the first end of the thick region is joined to the first end of the first thin region,
wherein the height of the thick region is greater than the height of the first thin region,
wherein the width of the thick region is greater than the width of the first thin region.

4. The system for safely storing sharp or hazardous materials of claim 3, wherein the first wall and the second wall each comprise:
a second thin region including a first end, a second end, a height, and a width,
wherein the second end of the thick region is joined to the second end of the second thin region,
wherein the height of the thick region is greater than the height of the second thin region,
wherein the width of the thick region is greater than the width of the second thin region.

5. The system for safely storing sharp or hazardous materials of claim 4, wherein the first wall and the second wall each comprise:
a first end cap joined to the second end of the first thin region; and
a second end cap joined to the first end of the second thin region,
wherein a height of the first end cap and a height of the second end cap are equal.

6. The system for safely storing sharp or hazardous materials of claim 5, wherein each of the thin regions comprise:
an inner surface connected to an outer surface by a top surface, wherein the top surface is perpendicular to the inner surface and the outer surface; and
a curved surface connecting the outer surface of each of the thin regions to the second surface of the floor.

7. The system for safely storing sharp or hazardous materials of claim 6, wherein the first wall and the second wall each comprise a single and contiguous inner surface spanning the first end cap, the first thin region, the thick region, the second thin region, and the second end cap along a single plane.

8. The system for safely storing sharp or hazardous materials of claim 2, wherein the first wall comprises a first recess and the second wall comprises a second recess.

9. The system for safely storing sharp or hazardous materials of claim 8, wherein the system further comprises an elongated restrictive element, wherein the elongated restrictive element spans across the channel and a first portion of the elongated restrictive element is positioned within the first recess and a second portion of the elongated restrictive element is positioned within the second recess.

10. The system for safely storing sharp or hazardous materials of claim 9, wherein the elongated restrictive element forms a contiguous loop and is in physical contact with at least two curved surfaces.

11. The system for safely storing sharp or hazardous materials of claim 10, wherein the elongated restrictive element comprises an elastic band.

12. The system for safely storing sharp or hazardous materials of claim 11, wherein the elongated restrictive element comprises a rigid material.

14. The system for safely storing sharp or hazardous materials of claim 1, wherein the second portion of the housing further comprises:
a planar element having an interior surface and an opposing exterior surface bounded by a perimeter; and
a wall projecting away from the planar element and joined to the perimeter of the planar element.

15. The system for safely storing sharp or hazardous materials of claim 14, wherein the first portion of the housing further comprises:
a planar element having an interior surface and an opposing exterior surface bounded by a perimeter; and
a wall projecting away from the planar element and joined to the perimeter of the planar element.

16. The system for safely storing sharp or hazardous materials of claim 15, wherein the wall of the second portion comprises:
a thick region having an exterior surface and an interior surface, wherein the interior surface of the thick region meets an interior surface of the planar element at a seam;
a thin region joined to the thick region, wherein the thin region has an exterior surface and shares the interior surface with the thick region; and
a lip connecting the exterior surface of the thick region with the exterior surface of the thin region,
wherein a top surface joins the interior surface with the exterior surface of the thin region.

17. The system for safely storing sharp or hazardous materials of claim 16, wherein the wall of the first portion comprises:
a thick region having an exterior surface and an interior surface, wherein the interior surface of the thick region meets an interior surface of the planar element at a seam;
a thin region joined to the thick region, wherein the thin region has an interior surface and shares the exterior surface with the thick region; and
a lip connecting the interior surface of the thick region with the interior surface of the thin region,
wherein a top surface joins the interior surface of the thin region with the exterior surface.

18. The system for safely storing sharp or hazardous materials of claim 17, wherein the lip of the second portion abuts the top surface of the first portion and the exterior surface of the thin region of the second portion abuts the interior surface of the thin region of the first portion.

19. The system for safely storing sharp or hazardous materials of claim 18, further comprising a groove extending along the lip of the second portion, wherein a sealing element resides within the groove.

20. The system for safely storing sharp or hazardous materials of claim 19, wherein the sealing element comprises an O-ring.

21. The system for safely storing sharp or hazardous materials of claim 20, wherein the structural element is coupled to the planar element of the second portion of the housing.

22. The system for safely storing sharp or hazardous materials of claim 21, wherein the structural element is coupled to the planar element of the second portion of the housing using one or more clips, a weld, or an adhesive.

23-39. (canceled)
